(19) United States
(12) Plant Patent Application Publication
(10) Pub. No.: US 2003/0115649 P1
(43) Pub. Date: Jun. 19, 2003

(54) Kerria japonica named 'Fubuki Nishiki'

(76) Inventor: Yuji Suzuki, Kawaguchi City (JP)

Correspondence Address: CHRISTIE, PARKER & HALE, LLP, 350 WEST COLORADO BOULEVARD, SUITE 500, PASADENA, CA, 91105, US

(21) Appl. No.: 10/027,597

(22) Filed: Dec. 19, 2001

(51) Int. Cl. A01H 5/00

(52) U.S. Cl. PLT/226

(57) ABSTRACT

A new plant variety of Kerria japonica characterized by White flecked variegated foliage and White and Creamy striped flowers.

### BACKGROUND OF THE INVENTION

The present invention relates to a new and distinct variety of Kerria japonica (L.) DC [Corchorus japonicus Thunb.], which was discovered as a branch sport from the species Kerria japonica in Saitama Prefecture, Japan. The varietal denomination of the new variety is ‘FUBUKI NISHIKI’.

The genus Kerria is included in the family Rosaceae, which comprises about 100 genera and 2,000 species of herbs, shrubs and trees native mostly to China and Japan. Kerria comprises one species, Kerria japonica (L.) DC, native to the temperate regions of eastern Asia, which possesses desirable ornamental characteristics.

Kerria japonica is a deciduous shrub with Green leaves and glabrous branches. Leaves of the typical species are alternate, oval, long acuminate, serrated on the margins with pinnate venation. It generally has Yellow flowers.

### SUMMARY OF THE INVENTION

The new variety was discovered as a branch sport and differs from its parent by white flecked variegated foliage and White and Creamy striped flowers. Asexual reproduction of the new variety by vegetative cuttings, performed in Lewisberry, Pa. and Vacaville, Calif. have confirmed that the distinctive characteristics of the new variety are stable and transmitted to succeeding generations, and the new variety reproduces true to type.

### COMPARISON WITH PARENTS

‘FUBUKI NISHIKI’ is distinguished from its parent and all other varieties of Kerria japonica of which I am aware by White flecked variegated foliage and White and Creamy marked flowers. The parent plant has completely Green foliage and Yellow flowers.

### BRIEF DESCRIPTION OF THE ILLUSTRATIONS

The accompanying illustrations show a specimen of the new cultivar in a photo illustration as true to color as is reasonably possible to make in an illustration of this character.

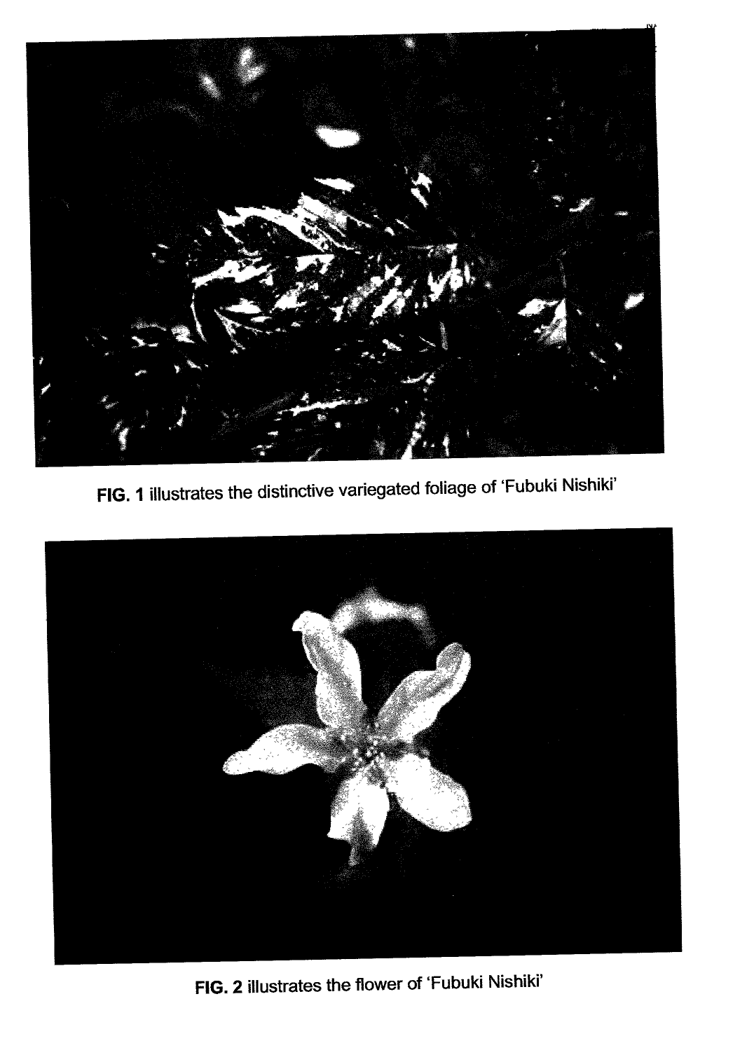

FIG. 1 illustrates the distinctive variegated foliage of ‘FUBUKI NISHIKI’; and

FIG. 2 illustrates the flower of ‘FUBUKI NISHIKI’.

### DETAILED DESCRIPTION OF THE INVENTION

‘FUBUKI NISHIKI’ has not been observed under all possible environmental, cultural and light conditions. The following observations and descriptions are of plants grown in Vacaville, Calif. and Lewisberry, Pa. In this description, color references are to The Royal Horticultural Society Colour Chart (1995) and terminology used in the color descriptions herein refers to plate numbers in this color chart. Phenotypic expression may vary with light intensity, cultural and environmental conditions.

Botanical Classification: Kerria japonica (L.) DC. [Corchorus japonicus Thunb.]

Parentage.—Branch sport from the species.

Propagation.—Stem cuttings.

Plant:

Size.—About 2 m tall; about 3 m across.

Habit.—Upright; multi-branched shrub when young, becoming spreading with age.

Branching.—Lateral Branches: Numerous, obliquely upright becoming pendant with age Typical Branch Number: More than 300 Observed Branch Number: 125 Size Length: About 2 m long Diameter: Between 4 to 5 mm Color New Shoots: Green Group, near 139C Mature Branches: Green Group, near 139C

Leaf:

Shape.—Ovate to oblong; leaf tip long; acuminate occasionally hooked; leaf base mostly cuneate to obtuse and occasionally cordate or truncate.

Size.—Between 9 to 10 cm long and about 5 cm wide.

Arrangement.—Alternate.

Margin.—Coarsely and irregularly doubly serrate.

Texture/Substance.—Thin, chartaceous Upper Surface: Glabrous with impressed veins, nearly bullate Under Side: Glabrous to slightly pubescent with prominent veins.

Color:

The following Color Readings are of specimens grown in Vacaville, Calif., USA and Lewisberry, Pa., 2 year old specimen in 5 gallon containers.

Variegation Pattern:

Flecked, splashed, and mottled in a random pattern. Areas of variegation are frequently long and narrow, parallel to the side veins of the leaf. There are additional smaller areas of variegation that show no consistent shape and are randomly distributed across the leaf. The variegation typically covers 25% to 50% of the leaf surface. Although the pattern of variegation is random, every leaf displays noticeable patterns of variegation. Foliage holds the initial variegation from emergence through the growing season.

Young foliage.—Green Sections Upper Side: Green Group, near 143A Under Side: Green Group, near 143B Variegated Sections Upper Side: Greyed-Green Group, near 196C and 196D and White Group, near 155B Under Side: Greyed-Green Group, near 196D.

Mature foliage.—Green Sections Upper Side: Green Group, near 143A Under Side: Green Group, near 143B Variegated Sections Upper Side: Greyed-Green Group, near 196C and 196D and White Group, near 155B Under Side: Greyed-Green Group, near 196D

Venation pattern.—Alternately pinnate depressed above, raised beneath.

Petiole.—Size: Between 0.5 to 1.5 cm Coloration: Green Group, near 143A Texture: Smooth Internodes: Between 1 to 8 cm.

Hardiness.—USDA Zone 4 (−30° F. to −20° F.).

Vigor.—Rapid growth rate.

Pests/Diseases.—No pest or disease problems noted.

Inflorescence:

Bloom period.—Outdoors: March to May Greenhouse: Not observed in greenhouse conditions.

Flower.—Rotate Arrangement: Solitary, terminal on lateral branchlets Size: About 3.5 cm wide.

Inflorescence shape.—Solitary, terminal on lateral branchlets.

Fragrance.—None noted.

Lastingness of individual blooms.—Between 3 to 5 days.

Petalage.—Number: About 5 Shape: Ovate Apex: Retuse occasionally apiculate Base: Abruptly attenuate Size: About 16 mm long; about 7 mm wide Margin: Entire and smooth, often undulate Curvature: Slightly inwardly cupped Arrangement: Rotate Color Ground color: Yellow-Orange Group, near 145B Variegated section: Greyed-Green Group, near 196C-D with occasional Yellow-Orange Group, near 11B; occasional random markings or stripes parallel to the long axis of the petal, occasional marginal variegation Sepals: Pointed, with some striped variegation.

Reproductive Organs:

Androecium.—

Stamens.—Between 30-40.

Anther.—Shape: Club shaped Color: Yellow-Orange Group, near 11B

Pollen amount.—Numerous Color: Yellow-Orange Group, near 11B.

Gynoecium.—Pistil count: About 5 Length: About 2 mm Stigma Color: Yellow-Orange Group, near 11B Style Color: Yellow-Orange Group, near 11B Ovary Color: Yellow-Orange Group, near 11B.

Fruit type.—Achene.

I claim:

1. A new plant variety of Kerria japonica of the variety substantially as shown and described.
